(19) United States
(12) Patent Application Publication
(10) Pub. No.: US 2017/0156861 A1
(43) Pub. Date: Jun. 8, 2017

(54) Percutaneous or Minimally Invasive Cardiac Valve Repair System and Methods of Using the Same

(71) Applicant: LC THERAPEUTICS, INC., Sacramento, CA (US)

(72) Inventors: James Longoria, Sacramento, CA (US); Roy Chin, Pleasanton, CA (US)

(21) Appl. No.: 15/318,325

(22) PCT Filed: Oct. 23, 2014
(86) PCT No.: PCT/US14/61951

§ 371 Date: Dec. 12, 2016

Related U.S. Application Data
(60) Provisional application No. 61/894,844, filed on Oct. 23, 2013.

(51) Int. Cl. A61F 2/24

(52) U.S. Cl. CPC A61F 2/2457; A61F 2/2466; A61B 17/0401

(57) ABSTRACT

Percutaneous or minimally invasive systems configured to deliver a synthetic chord to an internal body location are provided. Aspects of the percutaneous or minimally invasive systems include a synthetic chord present in a percutaneous minimally invasive delivery device. The systems and methods of the invention find use in a variety of applications, such as cardiac valve, e.g., mitral valve, repair.

### CROSS-REFERENCE TO RELATED APPLICATIONS

Pursuant to 35 U.S.C. §119 (e), this application claims priority to the filing date of the U.S. Provisional Patent Application Ser. No. 61/894,844, filed Oct. 23, 2013, the disclosure of which is incorporated herein by reference.

### INTRODUCTION

The mitral valve is composed of two leaflets attached to the mitral valve annulus, which are supported at the free edge by chordae tendineae (chords) attached to the inside wall of the left ventricle and to the papillary muscles. However, sometimes one or both of the valve leaflets become loose, due to loosening or failure of one or more of these chords. The valve then prolapses, and the seal that it normally provides between the left atrium and left ventricle becomes compromised, causing the blood to flow back into the left atrium during systole.

A variety of methods have been described for placement of artificial chordae tendineae to correct mitral valve leaflet prolapse and treat diseased mitral valve chordae tendineae. However, there are many technical challenges in this interventional procedure, especially when performed with minimally invasive techniques, particularly via the percutaneous approach. The most common method of repairing the valves is to create synthetic chordae tendineae from expanded polytetrafluoroethylene (ePFTE), which are fastened into place between the papillary muscle of the heart wall and the mitral valve leaflets. Cardiac surgeons usually are required to perform the time-consuming process of measuring and cutting the necessary length of synthetic chordae tendineae material during the surgical procedure after they have measured the dimensions of the patient's heart valves. In addition, anchoring the synthetic chordae tendineae in the papillary muscle and securing the fasteners through the leaflets is often technically difficult in minimally invasive procedures, because of limitations in using 2-dimensional video for viewing the surgical field, limited exposure of the surgical field, and limited degrees of freedom using standard thoracoscopic instrumentation.

### SUMMARY

Percutaneous or minimally invasive systems configured to deliver a synthetic chord to an internal body location are provided. Aspects of the minimally invasive systems include a synthetic chord present in a minimally invasive delivery device. The systems and methods of the invention find use in a variety of applications, such as cardiac valve, e.g., mitral valve, repair.

### BRIEF DESCRIPTION OF THE FIGURES

FIGS. 1A and 1B provide a view of a synthetic chord device in accordance with an embodiment of the invention, where the device is shown before and after deployment, respectively. FIGS. 1C and 1D provide a view of a synthetic chord device in accordance with another embodiment of the invention, where the device is shown before and after deployment, respectively.

FIG. 2 provides a schematic view of the normal left side of the heart.

FIG. 3 provides a schematic view of the left side of the heart demonstrating a ruptured chorda tendineae of the mitral valve.

FIGS. 4 to 7 depict a procedure for repairing a ruptured chorda tendineae using a catheter-based percutaneous or minimally invasive system according to an embodiment of the invention.

### DEFINITIONS

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, nervous tissue and/or epithelial tissue.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”).

The present disclosure provides embodiments of devices (e.g., a synthetic chord device or a portion thereof) that are implantable. As used herein, the terms “implantable”, “implanted” and “implanting” refer or relate to the characteristic of the ability of an aspect to be placed (e.g., interventionally introduced or surgically introduced) into a physiological site (e.g., a site within the body of a subject) and maintained for a period of time without substantial, if any, impairment of function. As such, once implanted in or on a body, the aspects do not deteriorate in terms of function, e.g., as determined by ability to perform effectively as described herein, for a period of 2 days or more, such as 1 week or more, 4 weeks or more, 6 months or more, or 1 year or more, e.g., 5 years or more, up to and including the remaining lifetime or expected remaining lifetime of the subject or more. Implantable devices may also be devices that are configured (e.g., dimensioned and/or shaped) to fit into a physiological site (e.g., a site within the body of a subject). For example, in certain embodiments, an implantable device may have a longest dimension, e.g., length, width or height, ranging from 0.05 mm to 150 mm, such as from 0.1 mm to 10 mm, including from 0.5 mm to 5 mm. Implanting may also include securing an implanted object (e.g., a prosthetic device) to one or more tissues within the body of the subject. Additionally, implanting may, in some instances, include all of the surgical procedures (e.g., cutting, suturing, sterilizing, etc.) necessary to introduce one or more objects into the body of a subject.

In some instances, the devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue piercing device enters tissue). Similarly, the term “distal” refers to a direction oriented away from the operator during use or a position (e.g., a spatial position) further from the operator (e.g., closer to a subject or tissue thereof) during use (e.g., at a time when a tissue piercing device enters tissue). Accordingly, the phrase “proximal end” refers to that end of the device that is closest to the operator during use, while the phrase “distal end” refers to that end of the device that is most distant to the operator during use.

Furthermore, the definitions and descriptions provided in one or more (e.g., one, two, three, or four, etc.) sections of this disclosure (e.g., the “Descriptions”, “Devices”, “Methods” and/or “Kits” sections below) are equally applicable to the devices, methods and aspects described in the other sections.

### DETAILED DESCRIPTION

Percutaneous or minimally invasive systems configured to deliver a synthetic chord to an internal body location are provided. Aspects of the percutaneous or minimally invasive systems include a synthetic chord present in a percutaneous or minimally invasive delivery device. The systems and methods of the invention find use in a variety of applications, such as cardiac valve, e.g., mitral valve, repair.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Percutaneous or Minimally Invasive Systems

As summarized above, aspects of the invention include percutaneous or minimally invasive systems that are configured to connect or align tissues, or connect tissue to a prosthesis, or a combination thereof. Systems as described herein may be configured to secure a papillary muscle to a valve leaflet, such as a mitral valve leaflet or tricuspid valve leaflet. When an aspect (e.g., a tissue, such as a valve leaflet) is secured, it may, for example, be retained at the same position or substantially at the same position (e.g., a position within the body of a subject) for a time period, such as a for a period of days, weeks, months, years and/or for at least the remaining lifetime of a subject.

As the systems are percutaneous or minimally invasive systems, they are configured for use in minimally invasive interventional or surgical applications. By “minimally invasive interventional or surgical application” is meant a procedure that is less invasive than an open surgical procedure. By “minimally invasive interventional or surgical application” is meant a procedure that is performed on a beating heart. A minimally invasive interventional or surgical procedure may involve the use of arthroscopic and/or laparoscopic devices and/or remote-control manipulation or catheter-based of interventional instruments and/or percutaneous devices. In some instances, the minimally invasive system is a percutaneous system. The term “percutaneous” refers to any medical procedure where access to inner organs or other tissue is done via needle-puncture of the skin, rather than by using an “open” approach where inner organs or tissue are exposed (typically with the use of a scalpel). Minimally invasive interventional or surgical procedures include endovascular procedures, which may be totally endovascular procedures, percutaneous endovascular procedures, etc. Endovascular procedures are procedures in which at least a portion of the procedure is carried out using vascular access, e.g., arterial or venous access. Minimally invasive interventional or surgical procedures also include open or endoscopic procedures, in which a device in inserted into the heart of patients to repair heart valve which may be on a beating heart.

As summarized above, systems of the invention include both a synthetic chord and a percutaneous or minimally invasive delivery device that is configured to deliver the synthetic chord to an internal body location, e.g., a cardiac location, such as described in greater detail below.

### Synthetic Chords

Synthetic chord devices as described herein include a flexible connector having a tissue securing member located at each end. The flexible connector has a first end and a second end. Embodiments of the synthetic chord devices include a first securing member at the first end of the first flexible connector. In some embodiments, the first securing member attaches the first end of the flexible connector to a tissue location following deployment of the securing member, e.g., as described in greater detail below. At the second end of the flexible connector is a second securing member. Various aspects of the embodiments of the devices, including the flexible connector, the first securing member and the second securing member, are now described in greater detail below.

### Flexible Connector

A synthetic chord device of certain embodiments of the subject invention includes a synthetic, or artificial, flexible connector, such as a flexible cord, line, filament, etc., which has first and second securing members at either end for attaching the connector to a tissue. In some embodiments, the flexible connector is configured to be attached to a prosthesis, or to a device that substitutes for or supplements a missing or defective part of the body, e.g., a synthetic cardiac valve, or a porcine valve. In some embodiments, a synthetic chord is configured to be used as a synthetic chorda tendineae for use in repair of a cardiac valve, e.g., the mitral valve.

The flexible connector (e.g., the first flexible connector) element is a flexible elongated structure having a first end and a second end. The first and second ends of the first flexible connector are not connected (e.g., do not form a continuous body of material or adjoin). As such, the first flexible connector does not form (e.g., is not shaped as) a loop (e.g., a continuous loop of one or more materials). In certain embodiments, the first flexible connector is constructed of one or more materials suitable for use in the body and that can be used in the methods of the subject invention, e.g., attaching a valve leaflet to the underlying cardiac tissue (e.g., attaching for an extended period of time, such as for the lifetime of the subject, without breaking). In some embodiments, the flexible connector does not include a knot. By “knot” as used herein is meant an interlacement (e.g., looping) or entanglement of portions of a body (e.g., a flexible connector) that forms a knob or lump. In some aspects, a knot prevents a body (e.g., a longitudinal, round body, such as a cord) having the knot from traveling through an opening in an aspect having an area that is slightly larger than the cross sectional area of the body. In some aspects, a knot is created by tying (e.g., purposefully tying) a body into an interlaced configuration. The flexible connector may be made up of a single line or filament, e.g., thread, or two or more such lines, which may where desired by twisted about each other, e.g., as present in a yarn.

The first flexible connector element has a length (e.g., length between the first and second end) suitable for extending from a first tissue to a second tissue, such that the flexible connector may be secured to both the first and the second tissue. In some embodiments, the flexible connector element has a length suitable for extending from a first tissue (e.g., a papillary muscle) to where it is secured to a second tissue (e.g., a mitral valve leaflet). The length of the first flexible connector may vary, and in some instances ranges from 5 mm to 100 mm, such as from 8 mm to 40 mm, including 10 mm to 30 mm. In some embodiments, the first or second end of the first flexible connector can be secured to a prosthesis, or other device that substitutes for or supplements a missing or defective part of the body, e.g., a synthetic cardiac valve, or a porcine valve, which is located at the target tissue location.

The flexible connector (e.g., the first flexible connector) can be made of a variety of materials. Such materials may be flexible materials. By “flexible”, as used herein is meant pliable or capable of being bent or flexed repeatedly (e.g., bent or flexed with a force exerted by a human hand or other body part) without damage (e.g., physical deterioration). A flexible material may be a material that remains able to perform intended function (e.g., repeatedly flexing) by remaining pliable for at least the expected lifetime or useful lifetime of the aspect which the material is included in. In some embodiments, the flexible connector may include biocompatible materials. The phrase “biocompatible materials” are materials that can be placed on or in living tissue for an extended period of time, such as for a period of 2 days or more, such as 1 week or more, 4 weeks or more, 6 months or more, or 1 year or more, e.g., 5 years or more, up to and including the remaining lifetime or expected remaining lifetime of the subject or more, and not cause a significant adverse (e.g., detrimental to health) reaction (e.g., an immune response) in the tissue or the associated organism.

Biocompatible materials, as included in the subject devices, can include any suitable biocompatible material, which material may or may not be biodegradable. Biocompatible materials of the subject devices, in some instances, are polymeric materials (e.g., materials having one or more polymers) and/or metallic materials. Such materials may have characteristics of flexibility and/or high strength (e.g., able to withstand significant force, such as a force exerted on it by a tissue within a human body, without breaking and/or resistant to wear) and/or high fatigue resistance (e.g., able to retain its physical properties for long periods of time regardless of the amount of use or environment). Biocompatible materials may also include any of the shape memory materials listed herein, as described in greater detail below.

In some embodiments, biocompatible polymeric materials of the subject devices, include, but are not limited to: polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, and combinations or mixtures thereof. Similarly, in certain embodiments, biocompatible metallic materials of the subject devices, include, but are not limited to: stainless steel, titanium, a nickel-titanium (NiTi) alloy (e.g., nitinol), a nickel-cobalt alloy, such as ELGILOY® cobalt-chromium-nickel alloy, tantalum, and combinations or mixtures thereof.

In certain embodiments, an active agent may be included in the composition of a biocompatible material, such as a polymeric material. As used herein, the phrase “active agent” refers to one or more chemical substances that, when administered to (e.g., placed in contact with or ingested by) a human, have one or more physiological effects. In some embodiments, the one or more active agents include an antithrombotic substance and/or an antibiotic substance and/or an anti-inflammatory (e.g., a substance that reduces or prevents inflammation). In various embodiments, a first flexible connector may be coated with a polymer, such as a polymer that releases one or more active agents (e.g., an anticoagulant that thereby reduces the risk of thrombus formation).

The cross-sectional configuration of the first flexible connector can be any suitable shape, such as round, oval, rectangular, square, etc. In some instances, the first flexible connector may have a flattened cross-sectional shape, such as a “ribbon” shape. In other embodiments, the flexible connector may be a combination of shapes, such as for example, a flexible connector that is round on two sides with a flat surface on the opposing two sides. In some embodiments the entire flexible connector has the same shape, and in other embodiments, at least a portion of the flexible connector may have a different shape, e.g., a ribbon configuration, or at least a portion of the connector that is flattened, or has a flat surface.

In some embodiments, the greatest outer diameter of the flexible connector ranges from 0.1 mm to 1.0 mm, such as from 0.1 mm to 0.5 mm, or 0.15 mm to 0.25 mm. In some embodiments, the entire flexible connector has the same diameter. In other embodiments, at least a portion of the connector has a different diameter, e.g., a smaller diameter. In some embodiments, at least a portion of the connector may have both a different configuration and a different diameter, e.g., a portion of the connector may have a flat surface, where the portion of the connector having a flat surface has a largest outer diameter larger than the remainder of the connector.

### First Tissue Securing Member

The synthetic chord devices further include a first tissue securing member located at an end (e.g., the first end) of a flexible connector. The first tissue securing member is configured to attach a flexible connector (e.g., a first flexible connector), such as those described above, to a tissue, e.g., a papillary muscle, as desired.

The first tissue securing member is a component configured to secure first end of a flexible connector to a target tissue location, (e.g., a papillary muscle or mitral valve, depending on the particular interventional or surgical protocol that is employed). In some embodiments, the first securing member of a synthetic chord device is located at, and/or attached to the first end of a first flexible connector of the device. By “secure” is meant that the securing member provides for stable association of the end of the flexible connector to the target tissue location, e.g., papillary muscle or mitral valve leaflet. By “stable association” is meant that the end of the flexible connector is substantially if not completely fixed relative to the tissue location of interest such that when the end of the flexible connector moves, the target tissue location to which it is secured by the deployed securing member also moves.

An aspect of the first securing members as described herein is that the securing member transitions from a linear to a planar configuration upon deployment, e.g., as described in greater detail below. As such, following initial placement systemic delivery device at the desired anatomical location, deployment of the synthetic chord, e.g., the first end of the synthetic chord, results in a change in configuration of the first securing member from a linear to planar configuration.

In some instances, deployment of the securing member results in an increase of the amount that is occupied by the securing member of a theoretical plane at least substantially perpendicular to the longitudinal axis of the flexible connector. The at least substantially perpendicular theoretical plane is a theoretical plane that is completely perpendicular to the longitudinal axis of the flexible connector, or at least closer to perpendicular than parallel, and in some instances is one that is at an angle ranging from 45° to 90° relative to the longitudinal axis of the flexible connector. The increase in the amount of the theoretical plane that is occupied by the securing element upon deployment may vary, and in some instances the magnitude of the increase is 5% or more, such as 10% or more, including 25% or more, e.g., 50% or more, up to 100% or more, and in some instances ranges from 5 to 5000%, such as 10 to 2500%.

Upon deployment, the planar configuration may be configured to cover a surface of the tissue sufficient to secure the first end of the flexible connector to the tissue, e.g., such that the first end can no longer be pulled through the tissue via the tissue passageway occupied by the first end of the flexible connector. In some instances, the surface area of the tissue covered by the securing member upon deployment into a planar configuration ranges from 0.05 mm² to 50 mm², such as 2 mm² to 25 mm², e.g., 5 mm² to 20 mm².

In some instances, the securing member has a low-profile upon deployment. By “low-profile” is meant that the top of the securing member when deployed does is not located at a substantial height relative to the surface of the target tissue to which it is secured. While the height of a given low profile securing element may vary, in some instances the height ranges from 0.05 to 5 mm, such as 0.1 to 2 mm, e.g., 0.2 to 1 mm, above the surface of the target tissue to which it is secured.

In some embodiments, the pre-deployment linear configuration is one that lacks a secondary structure, such that it appears in only a single location, e.g., as a small circle or dot (e.g., having a longest cross-sectional dimension (such as a diameter) ranging in some instances from 0.1 mm to 1.0 mm), in any cross-sectional plane passing through the securing member along the length of the securing member. As such, the pre-deployment linear configuration may be viewed as a one-dimensional configuration. The post-deployment planar configuration is one in which the securing member has a secondary configuration, such that there exists one or more cross-sectional planes passing through the securing member along the length of the securing member where the securing member is present at two or more locations. As such, the post-deployment planar configuration may be viewed as a two- or three-dimensional configuration, depending on the particular embodiment. The first securing member may assume a variety of different planar configurations. These configurations may include any number of different curvilinear configurations, including but not limited to serpentine configurations, spiral (e.g., disc-shaped) configurations, etc. The area defined by the planar configuration may vary so long as it is sufficient to secure the end of the first flexible member to the tissue location of interest, and in some instances ranges from 0.05 mm² to 50 mm², such as 2 mm² to 25 mm², e.g., 5 mm² to 20 mm², and in some embodiments ranges from 0.5 to 25 mm², such as 1 to 20 mm², including 1 to 10 mm².

In yet other embodiments, the pre-deployment linear configuration is one that transitions upon separation and deployment from: (a) a first configuration in which it has a longitudinal axis that is at least substantially parallel to the longitudinal axis of the flexible connector (i.e., a longitudinal axis that is substantially if not completely parallel with the longitudinal axis of the flexible connector) to (b) a second configuration where it has a longitudinal axis that is at least substantially perpendicular (i.e., is substantially if not completely perpendicular) to the longitudinal axis of the flexible connector. An example of such a configuration is a bar-shaped securing member which is connected to the flexible connector in a manner sufficient to provide for the desired transition from first to second configuration upon deployment. While dimensions of bar shaped securing members may vary, in some instances the bars have a length ranging from 1 to 15 mm, such as 2 to 10 mm, e.g., 3 to 5 mm, a width ranging from 0.2 to 5 mm, such as 0.25 to 2.5 mm, e.g., 0.5 to 1 mm and a height ranging from 0.2 to 5 mm, such as 0.25 to 2.5 mm, e.g., 0.5 to 1 mm.

Prior to deployment from the delivery device, the securing member may or may not be retained in its linear configuration by one or more mechanical restraining devices, such as a body of material on or within the securing member. Since the securing member is biased to remain in a planar configuration, when the one or more mechanical restraining devices are removed from the securing member upon separation of the tissue piercing member therefrom, the securing member transitions from a linear configuration to a planar configuration. The securing member may be attached to the flexible connector using any convenient approach, e.g., by a loop of the flexible connector through a receiving hold of the securing member, by a clip attachment, or by any other convenient connector.

In some instances, the first tissue securing member includes an end (e.g., the end that is furthest from the flexible connector, i.e., the end that is not attached to the flexible connector) that is configured to pierce tissue. By configured to pierce tissue is meant that, upon contact with tissue, the end is configured to penetrate into or run through tissue. For example, the end of the first tissue securing member may be pointed or sharp, e.g., as is present at the end of a needle.

Devices as described herein and portions thereof (e.g., securing members) may be fabricated from any convenient material or combination of materials. Materials of interest include, but are not limited to: polymeric materials, e.g., plastics, such as polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, etc., metals and metal alloys, e.g., titanium, chromium, stainless steel, etc., and the like. In some embodiments, the devices include on or more components (e.g., securing members) made of a shape memory material. Shape memory materials are materials that exhibit the shape memory effect, where the materials that have a temperature induced phase change, e.g., a material that if deformed when cool, returns to its “undeformed”, or original, shape when warmed, e.g., to body temperature. Where desired, the shape memory material may be one with a transformation temperature suitable for use with a stopped heart condition where cold cardioplegia has been injected for temporary paralysis of the heart tissue (e.g., temperatures as low as 8-10 degrees Celsius). The shape memory material may also be heat activated, or a combination of heat activation and pseudoelastic properties may be used. Shape memory materials of interest include shape memory metal alloys, such as alloys of nickel (e.g., nickel titanium alloy (nitinol), nickel cobalt alloys (e.g., ELGILOY® cobalt-chromium-nickel alloy, etc.), zinc, copper (e.g., CuZnAl), gold, iron, etc. Also of interest are non-metallic materials that exhibit shaper memory qualities, e.g., shape memory plastics, etc.

### Second Securing Member

Located at a second end of the flexible connector is a second member. As with the first securing member, the second securing member may be an element which transitions from a linear to a planar configuration upon deployment. As such, prior to or following placement of the second end of the flexible connector at the target tissue site, a change in configuration of the second securing member from a linear to planar configuration occurs.

In some instances, deployment of the second securing member results in an increase of the amount that is occupied by the second securing member of a theoretical plane at least substantially perpendicular to the longitudinal axis of the flexible connector. The at least substantially perpendicular theoretical plane is a theoretical plane that is completely perpendicular to the longitudinal axis of the flexible connector, or at least closer to perpendicular than parallel, and in some instances is one that is at an angle ranging from 45° to 90° relative to the longitudinal axis of the flexible connector. The amount of the theoretical plane occupied by the second securing member that is increased upon deployment may vary, and in some instances the magnitude of the increase is 5% or more, such as 10% or more, including 25% or more, e.g., 50% or more, up to 100% or more, and in some instances ranges from 5 to 5000%, such as 10 to 2500%.

Upon deployment, the planar configuration may be configured to cover a surface of the tissue sufficient to secure the second end of the flexible connector to the target tissue, e.g., such that the second end can no longer be pulled through the tissue via the tissue passageway occupied by the second end of the flexible connector. In some instances, the surface area of the tissue covered by the reinforcing element upon deployment into a planar configuration ranges from 0.05 mm² to 50 mm², such as 2 mm² to 25 mm², e.g., 5 mm² to 20 mm².

In some instances, the second securing member has a low-profile upon deployment. By “low-profile” is meant that the top of the securing member when deployed is not located at a substantial height relative to the surface of the target tissue to which it is secured. While the height of a given low profile second securing member may vary, in some instances the height ranges from 0.05 to 5 mm, such as 0.05 to 2.5 mm, e.g., 1 to 2 mm, above the surface of the target tissue to which it is secured.

In some embodiments, the linear configuration of the second securing member is one that lacks a secondary structure, such that it appears in only a single location, e.g., as a small circle or dot (e.g., having a longest cross-sectional dimension (such as a diameter) ranging in some instances from 0.1 mm to 1.0 mm), in any cross-sectional plane passing through the securing member along the length of the securing member. As such, pre-deployed linear configuration may be viewed as a one-dimensional configuration. The post-deployed planar configuration is one in which the second securing member has a secondary configuration, such that there exists one or more cross-sectional planes passing through the securing member along the length of the securing member where the securing member is present at two or more locations. As such, the post-deployment planar configuration may be viewed as a two- or three-dimensional configuration, depending on the particular embodiment. The second securing member may assume a variety of different planar configurations. These configurations may include any number of different curvilinear configurations, including but not limited to serpentine configurations, spiral configurations, etc. The area defined by the planar configuration may vary so long as it is sufficient to secure the end of the first flexible member to the tissue location of interest, and in some instances ranges from 0.05 mm² to 50 mm², such as 2 mm² to 25 mm², e.g., 5 mm² to 20 mm², and in some embodiments ranges from 0.5 to 25 mm², such as 1 to 20 mm², including 1 to 10 mm².

In yet other embodiments, the pre-deployment linear configuration is one that transitions upon deployment from: (a) a first configuration in which it has a longitudinal axis that is at least substantially parallel to the longitudinal axis of the flexible connector (i.e., a longitudinal axis that is substantially if not completely parallel with the longitudinal axis of the flexible connector) to (b) a second configuration where it has a longitudinal axis that is at least substantially perpendicular (i.e., is substantially if not completely perpendicular) to the longitudinal axis of the flexible connector. An example of such a configuration is a bar shaped reinforcing element which is connected to the flexible connector in a manner sufficient to provide for the desired transition from first to second configuration upon deployment. While dimensions of bar shaped securing members may vary, in some instances the bars have a length ranging from 1 to 15 mm, such as 2 to 10 mm, e.g., 3 to 5 mm, a width ranging from 0.2 to 5 mm, such as 0.25 to 2.5 mm, e.g., 0.5 to 1 mm and a height ranging from 0.2 to 5 mm, such as 0.25 to 2.5 mm, e.g., 0.5 to 1 mm.

In some instances, the second securing member has the same structure as the first securing member. For example, the first and second securing members may both be components that transition from a first, linear configuration to a second, spiral configuration, upon deployment. In yet other embodiments, the first and second securing members may have difference configurations. For example, the second securing member may have the bar configuration, e.g., as described above, and the first securing member may have a configuration that transitions to a spiral configuration upon deployment. As mentioned above, deployment of the second securing member may occur before or after positioning of the second end of the flexible connector at the second target tissue site, and in some instances occurs upon deployment.

Devices as described herein and portions thereof (e.g., reinforcing elements) may be fabricated from any convenient material or combination of materials. Materials of interest include, but are not limited to: polymeric materials, e.g., plastics, such as polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, etc., metals and metal alloys, e.g., titanium, chromium, stainless steel, etc., and the like. In some embodiments, the devices include on or more components (e.g., securing members) made of a shape memory material. Shape memory materials are materials that exhibit the shape memory effect, where the materials that have a temperature induced phase change, e.g., a material that if deformed when cool, returns to its “undeformed”, or original, shape when warmed, e.g., to body temperature. Where desired, the shape memory material may be one with a transformation temperature suitable for use with a stopped heart condition where cold cardioplegia has been injected for temporary paralysis of the heart tissue (e.g., temperatures as low as 8-10 degrees Celsius). The shape memory material may also be heat activated, or a combination of heat activation and pseudoelastic properties may be used. Shape memory materials of interest include shape memory metal alloys, such as alloys of nickel (e.g., nickel titanium alloy (nitinol), nickel cobalt alloys (e.g., ELGILOY® cobalt-chromium-nickel alloy, etc.), zinc, copper (e.g., CuZnAl), gold, iron, etc. Also of interest are non-metallic materials that exhibit shaper memory qualities, e.g., shape memory plastics, etc.

### Additional Aspects

Additionally, embodiments of the disclosed devices or one or more portions thereof (e.g., a synthetic chord, one or more flexible connectors, and/or a reinforcing element) may be symmetrical with respect to one or more (e.g., one, two, or three) and/or only one or more planes. Such planes may be cross-sectional planes which include at least a portion of one or more device portions therein. Also, in some embodiments of the disclosed synthetic chord devices, the devices have a first end (e.g., an end at which a tissue piercing member is located) and a second end (e.g., an end at which a reinforcing element is located) and the first end of the device is not symmetrical with the second end.

Synthetic chords that find use in embodiments of the present invention are also described in PCT Application Serial Nos. PCT/US2014/040943 and PCT/US2014/048305; the disclosures of which applications are herein incorporated by reference.

### Specific Embodiments of Synthetic Chords

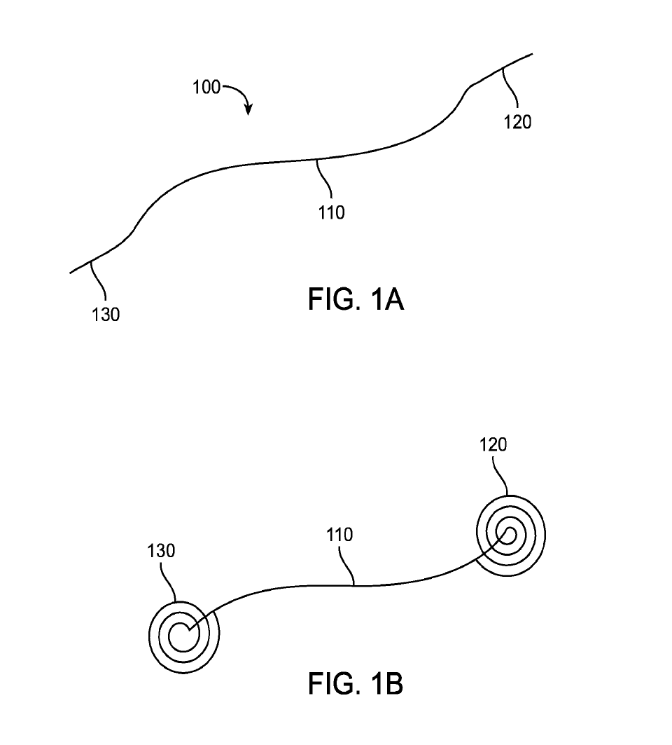

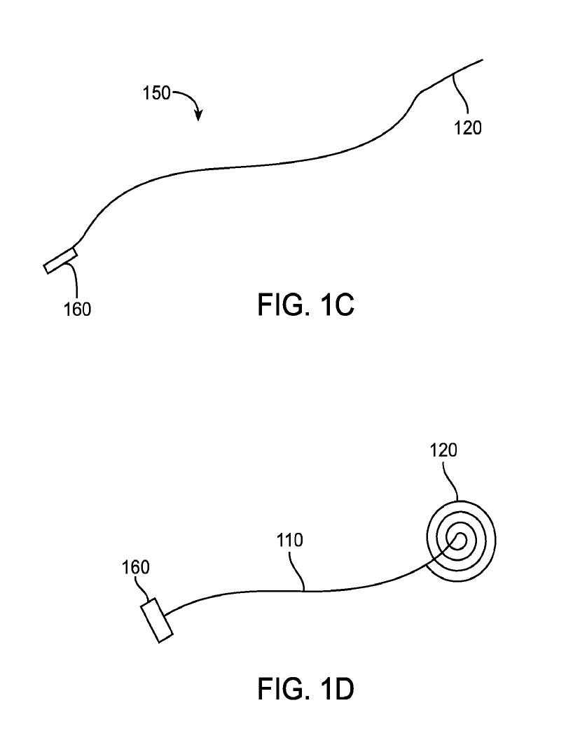

FIGS. 1A and 1B provide a view of a synthetic chord device which may be deployed by systems in accordance with an embodiment of the invention. In FIG. 1A, a synthetic chord device 100 is shown in an un-deployed state. The device includes flexible connector 110 having a first end connected to a first securing member 120 and a second end connected to a second securing member 130. When present in the percutaneous or minimally invasive delivery device, first and second securing members 120 and 130 are maintained in a restrained, linear state. In FIG. 1B, the synthetic chord device of the depicted embodiment of FIG. 1A is shown in a deployed state. The first and second securing members 120 and 130 have assumed an unconstrained, spiral planar configuration. The deployed securing members 120 and 130 assume a planar spiral configuration having an area sufficient to secure the end of the flexible member to the tissue location. Flexible connector 110 is also shown having a first end connected to the first securing member 120 and a second end connected to the second securing member 130. The device depicted in FIGS. 1A and 1B is an example of an embodiment where the first and second securing members each have a pre-deployment linear configuration that may be viewed as a one-dimensional configuration and a post-deployment planar configuration in which the securing member has a secondary configuration, as described in greater detail below.

FIG. 10 provides a view of the device in accordance with another embodiment of the invention. In FIG. 10, a synthetic chord device 150 is shown in an un-deployed state. The device is analogous to the device shown in FIGS. 1A and 1B, except that the linear/planar spiral second reinforcing member 130 has been replaced with a bar 160 which transitions from an un-deployed configuration in in which its longitudinal axis is parallel with that of the flexible connector 110 to a second deployed configuration, shown in FIG. 1D, in in which its longitudinal axis is perpendicular with that of the flexible connector 110. In the deployed state, shown in FIG. 1D, the second securing member 160 has assumed a second configuration, as shown, where its longitudinal axis is perpendicular with the longitudinal axis of the flexible connector 110. The deployed first and second securing members assume a configuration having an area sufficient to secure the end of the flexible member to the tissue location.

### Percutaneous or Minimally Invasive Delivery Device

As summarized above, systems as described herein further include a percutaneous or minimally invasive delivery device. By percutaneous or minimally invasive delivery device is meant a device configured to position or place a synthetic chord, e.g., as described above, at an internal body location via an interventional or minimally invasive procedure, such as a percutaneous procedure. The percutaneous or minimally invasive delivery device may have a variety of different configurations. For example, the device may be configured to access the target tissue location via a vascular route, via a trocar, etc. Minimally invasive devices of interest include, but are not limited to, endoscopic devices, catheter devices, etc.

While percutaneous or minimally invasive deployment devices may vary, in some instances the devices are catheter devices that include one or more passageways or lumens. Catheter delivery devices include a proximal end and a distal end separated by an elongated tube. By elongated, it is meant that the distance between the proximal and distal ends is sufficient for the catheter to be inserted or introduced into the vascular system of a patient at a site remote from the target tissue location that is to be manipulated upon deployment of the synthetic chord from the delivery device. Catheters intended for intravascular introduction may vary in length, and in some instances have a length in the range from 20 cm to 200 cm and an outer diameter in the range from 1 French (0.33 mm; Fr.) to 14 Fr., such as from 3 Fr. to 10 Fr. In the case of catheter delivery devices configured for delivery of a synthetic chord to a cardiac location, e.g., a mitral valve location, the length may range from 20 to 200 cm, and the outer diameter may be 20 Fr. or lower, such as 10 Fr. or lower, and in some instances may range from 6 Fr. to 10 Fr. In certain embodiments, the elongated tubular element has a length of from 20 to 200 cm, such as from 50 to 120 cm and including from about 60 to 100 cm.

The catheter delivery devices may include a multiport manifold at their proximal ends. By multiport manifold is meant a manifold that includes two or more ports (in addition to the attachment structure of the manifold to the proximal end of the elongated tube of the catheter), where the number of ports in the manifold may range from 2 to 4, depending on the particular catheter delivery device. The ports may be configured to receive various elements, e.g., guidewires, a deployment element actuator, etc. The tube may be fabricated from any convenient material, and in some instances is a polymeric extruded element, which is made up of one or more biocompatible polymers that have been extruded to produce the tube. Biocompatible polymers of interest include, but are not limited to: polyimide, polyamide, PBAX™, polyethylene, polyisoprene, nylon and the like.

The catheter device, at least at the distal end, may include an inner space configured to house a synthetic chord prior to deployment and an opening through which the synthetic chord may be deployed, e.g., through which the synthetic may be moved from its location in the device to the target tissue location outside of the device. While the dimensions of the inner space, i.e., compartment, that is configured to house the synthetic chord prior to deployment may vary, in some instances the compartment has a volume ranging from 20 to 400 mm³, such as from 40 to 300 mm³ and including from 70 to 200 mm³. The dimensions of the opening at the distal end of the catheter device may also vary so long as the dimensions are sufficient for the synthetic chord to be deployed through the opening, and in some instances the opening has a diameter ranging from 0.05 to 3 mm, such as from 0.1 to 2 mm and including from 0.2 to 1 mm.

Also located at the distal end of the catheter may be a deployment element configured to deploy the synthetic chord from the compartment through the opening to the target location. The deployment element may be any convenient device, which may be simple pushing device that controllably moves the synthetic chord by pushing from the compartment out the opening to the tissue location.

In some instances, the catheter device is a steerable catheter device. Various steerable mechanisms have been disclosed to steer catheters and other elongated medical devices, e.g., steerable guidewires and stylets, that involve use of a deflection mechanism extending through a deflection lumen of the catheter body to an attachment point in the catheter body distal segment. Typically, elongated wires variously referred to as control lines or reins or deflection wires or traction wires or push-pull wires or pull wires (herein “deflection wires” unless otherwise specified), extending between a proximal control mechanism and the distal attachment point. More complex steerable catheters have two or more deflection lumens and deflection wires extending from the handle through the is deflection wire lumens to different points along the length or about the circumference of the catheter body to induce bends in multiple segments of the catheter body and/or in different directions. The deflection lumens extend parallel to the central catheter body axis. In many cases, a handle is attached at the elongated catheter body proximal end, and the proximal end(s) of the deflection wire(s) is coupled to movable control(s) on the handle that the user manipulates to selectively deflect or straighten the distal segment and, in some cases, intermediate segments of the catheter body. Specifically steerable catheter configurations which may be readily adapted to deliver synthetic chord devices in accordance with aspects of the invention are described in U.S. Pat. Nos. 8,500,733; 8,394,091; 8,388,572; 8,376,990; 8,273,285; 7,959,601; 7,771,388; 7,717,875; 7,682,358; 7,608,056; 7,412,274; 7,232,422; 7,077,823; 7,037,290; 7,027,851 and 7,025,759; the disclosures of which are herein incorporated by reference.

### Methods

Synthetic chord devices, e.g., as described above, find use in methods for connecting a first tissue, such as a cardiac valve leaflet, to a second tissue, such as a papillary muscle. The subject devices therefore find use in methods in which a prolapsed cardiac valve leaflet, such as a mitral valve leaflet, is repaired. Methods for repair of a cardiac valve, such as a mitral valve, are discussed below. When performing a minimally invasive procedure, e.g., wherein the heart and heart valve are accessed through minimally invasive openings in the thoracic cavity, such as through trocar cannulas or small incisions in the intercostal spaces, or via a vascular approach, the minimally invasive procedures can be viewed remotely using a camera and monitor, or in some cases directly, as desired.

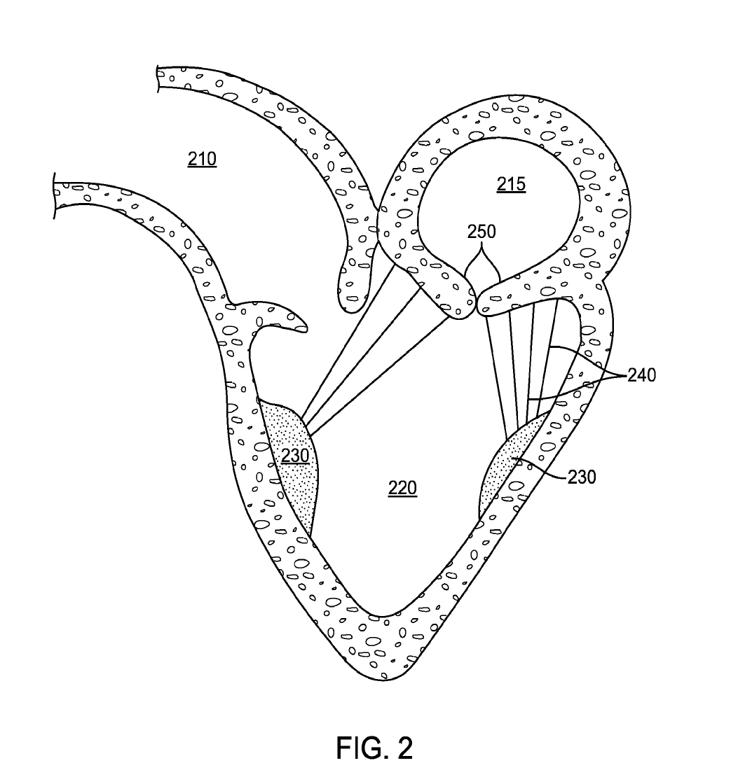

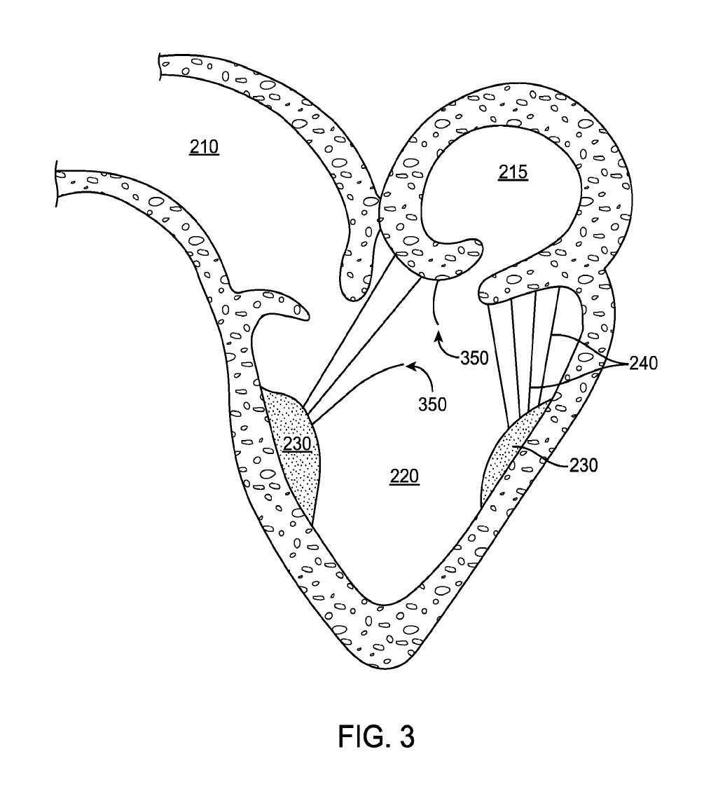

FIG. 2 depicts a schematic drawing of the left side of the heart. The aortic arch 210, left atrium 215, and left ventricle 220 are shown, with the mitral valve 250 located between the left ventricle and the left atrium. The chordae tendineae are shown as elements 240, attached to the leaflets of the mitral valve on one end, and the papillary muscle 230 in the left ventricle on the other end. An illustration of a rupture, or breakage of one of the chorda tendineae (350) that can be repaired using the methods and devices of the subject invention is shown in FIG. 3. FIG. 3 depicts a schematic drawing showing portions of the heart including the aortic arch 210, left atrium 215, and left ventricle 220, with the mitral valve 250 located between the left ventricle and the left atrium. The chordae tendineae are shown as elements 240, attached to the leaflets of the mitral valve on one end, and the papillary muscle 230 in the left ventricle on the other end. The ruptured, or broken chorda tendineae is shown as element 350. The leaflets of the mitral valve now no longer coapt, or close, and during systole, blood can flow from the left ventricle back into the left atrium, i.e., mitral regurgitation.

Prior to delivery of the synthetic chord, the desired length of the flexible connector is determined by measuring the distance between the prolapsed mitral valve leaflet and the papillary muscle using methods that are well known in the art. The desired length for the flexible connector can be determined using any suitable measuring device, such as a caliper, or a Mohr Suture Ruler Device™ (Geister, Tuttlingen, Germany). For example, a caliper or sterile disposable flexible tape measure can be used to assess the correct length for the synthetic mitral valve chordae by measuring the distance between the tip of the papillary muscle and the edge of a non-prolapsing segment of the mitral valve leaflet. The measurement can also be confirmed by comparison with pre-operative transesophageal echocardiography (TEE) in intra-operative 3D echocardiography. If a set of synthetic chord devices is provided, the synthetic chord device having a flexible connector with the desired length, or the closest to the desired length, is then selected from among the set of synthetic chord devices. The set of synthetic chord devices can include two or more first flexible connectors of the same or of different lengths, such as three connectors, or four connectors, etc. If a set of synthetic chord devices is not provided, but instead, an appropriate single synthetic chord device is available, that synthetic chord device is selected for use.

For deployment and implantation of the synthetic chord device, any convenient minimally invasive protocol and delivery device may be employed. Where a catheter based delivery system is employed, the distal end of the catheter may be advanced from a percutaneous vascular insertion site to the target tissue location, e.g., a cardiac location, such as the left ventricle or atrium. For example, a synthetic chord may be advanced via one or more catheters to the proximity of the prolapsed valve leaflet in an anterograde approach (e.g., from above the mitral valve). Alternatively, a synthetic chord device may be advanced via a retrograde approach (e.g., from below the mitral valve). In all of the methods described herein, the cardiac tissue located below the prolapsed valve (to which a reinforcing element is attached) may be selected from the group consisting of a papillary muscle and a ventricular wall. Also of interest are endoscopic based protocols, e.g., where a synthetic chord device is delivered via an endoscopic device, e.g., through a trocar, to a target location, such as described above.

In deploying the synthetic chord device from the delivery device, the first end of the chord device that includes the first securing member is moved out of the opening of the delivery device, e.g., out of an opening at the distal end of the delivery device, in a manner such that it passes through the target tissue locations to be connected by the chord, e.g., a mitral valve leaflet and a papillary muscle. To assist in passing the first securing member through the target tissue locations, the first securing member may include a sharpened end configured to pierce tissue, e.g., as described above. The first securing member is first passed (e.g., advanced) sequentially through the tissues to be connected, e.g., through a mitral valve leaflet and then through papillary muscle. To maintain the securing member(s) in constrained configured, a companion wire or analogous mechanical structure releasably associated with the chord may be employed to advance the chord from the delivery device. Upon passage of the first reinforcing member through both tissue locations to be connected, the first securing member assumes a second planar configure that secures the first end of the flexible connector to the last of the tissues that it has been through deployment may be assisted by removal of a securing means, e.g., restraining wire, such as described above. The delivery device may then be removed from the target location in a manner that deploys the second reinforcing member, such that the second tissue location is securedly connected to the first distal location.

During delivery of the chord device, the position of the prolapsed valve leaflet may be adjusted by coordinating the tension of the first flexible connector and the location of the leaflet, as desired. The valve leaflet position may be adjusted in real-time in a beating heart (e.g., using echocardiography). For example, the valve leaflet may be repositioned while monitoring mitral regurgitation (MR). Once any MR is reduced or eliminated, the valve leaflet is in the correct position. Once the valve leaflet is positioned correctly, the second securing member can then be deployed to transition the securing member to the planar configuration and thereby connect a second tissue (e.g., a cardiac valve leaflet) to a first tissue (e.g., a papillary muscle). It should be noted that the number of synthetic chord devices required to secure the connecting tissues together may vary depending on the procedure and the anatomy.

By this method, a prolapsed mitral valve leaflet can be repaired by securing the leaflet to the papillary muscle below. Using the methods and devices of the subject invention, a mitral valve repair procedure can be successfully completed without the need for the time-consuming step of cutting the desired length of synthetic cord while the patient is on the operating table, thereby decreasing the amount of time needed to place a patient on cardio-pulmonary bypass. In addition, the subject methods and devices obviate the need for tying sutures and ensuring that the suture material does not become tangled, difficulties which are exacerbated by the small size of the tissues involved and the often limited field of the operation.

Any appropriate prolapsed valve leaflet may be treated as described herein, including mitral valve leaflets and tricuspid valve leaflets. Further, these methods may be performed using one or more catheters or using non-catheter surgical methods, or using a combination of catheter-type surgical methods and non-catheter type surgical methods. The methods of the subject invention may also be used in combination with other surgical procedures, e.g. replacement of a mitral valve annulus, etc.

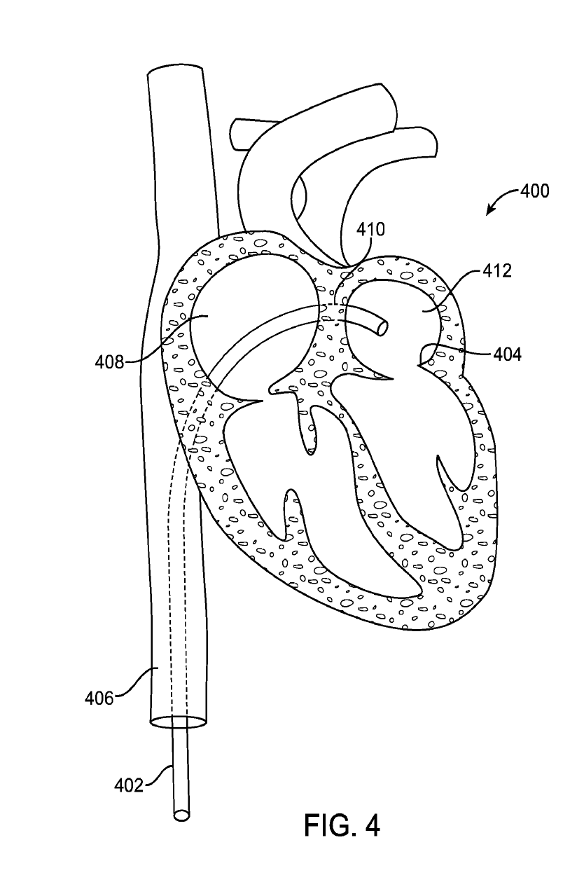

FIGS. 4 to 7 depict a percutaneous or minimally invasive procedure for repairing a ruptured chorda tendineae as depicted in FIG. 3, using a percutaneous or minimally invasive system that includes a catheter delivery device, e.g., as described above. FIG. 4 shows a cross-section of heart 400, having a prolapsed mitral valve leaflet 404. To access the prolapsed mitral valve leaflet 404, a trans-septal catheter 402 is introduced into the inferior vena cava 406, which is in turn accessed through one of the femoral veins. The trans-septal catheter 402 is then advanced up through the right atrium 408 and through the inter-atrial septum 410. Examples of the trans-septal approach are described in U.S. Pat. Nos. 6,743,239, 6,695,866 (herein incorporated by reference in their entirety). The catheter may be flexible and steerable, so that it can be maneuvered through the tortuous anatomy of the vasculature. For example, the catheter may include a steerable sheath, wherein at least part of the sheath (e.g., the distal end) is steerable in one or more directions, e.g., as described above. Thus, the sheath may be inserted trans-septally and oriented (e.g., towards the anterior commissure) so that another catheter may pass through the sheath and be further steered towards the valve leaflet and/or the cardiac tissue located beneath prolapsed valve leaflet.

FIGS. 4 to 7 depict one method of accessing the prolapsed mitral valve leaflet 404 (e.g., the anterograde approach), however different methods of access are also suitable. For example, a catheter may also be introduced via the jugular vein, may be introduced through the right femoral artery, and advanced up to the left atrium 412 by crossing the aortic valve, or may be introduced via the carotid or subclavian arteries. Thus, the order of the steps described in the method may be adapted to suit these variations. For example, in a retrograde approach the valve is accessed from below, and the distal end of the cord is attached to a first securing member adapted to secure to the valve leaflet, and the second securing member is adapted to secure to the cardiac tissue or papillary muscle located beneath prolapsed valve leaflet and be slideably connected to the cord.

Any appropriate visualization technique may be used to help the practitioner visualize the valve anatomy, and to manipulate or steer the catheters. For example, intracardiac echo, or transesophageal echo may be used or the 3D echo. In another visualization method, a laser fiberscope may be used to visualize target tissue in the blood pool. Thus, the devices described herein may be adapted to enhance visualization of the devices when used with any of the techniques. For example, the devices may include contrasting agents, and they may include electron dense or radioopaque regions, etc. In addition, rapid ventricular pacing, or adenosine IV administration may allow for transient and reversible cardiac arrest in order to stabilize the leaflets and papillary muscles and facilitate targeting.

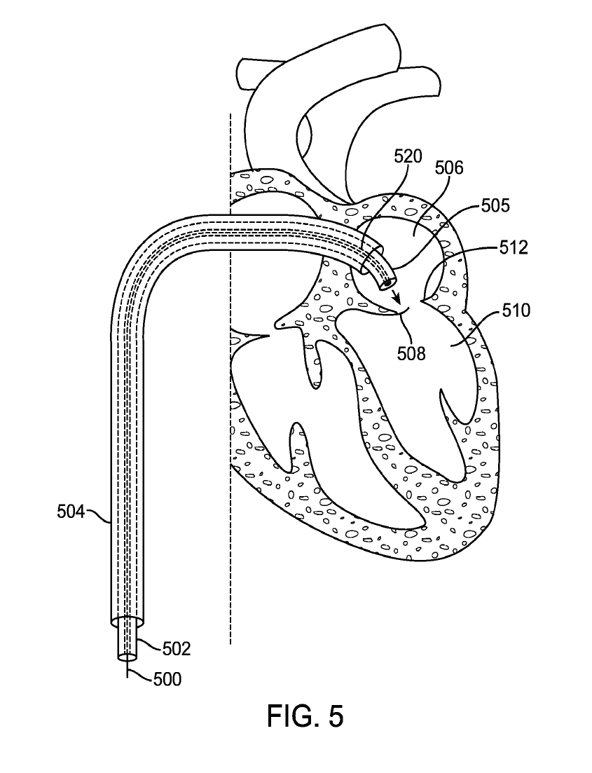

FIG. 5 provides a magnified view of the heart cross-section of FIG. 4. Shown in FIG. 5 is delivery catheter 502 introduced into trans-septal catheter 504 (e.g., a trans-septal sheath). Delivery catheter 502 is includes at its distal end 505 a synthetic chord device, 520, e.g., as depicted in FIGS. 1A to 1D. Also present is releasable restraining wire 502 which can be released from the chord device following placement at the target tissue location in order to deploy the chord and secure the tissue locations to each other. Delivery catheter 502 is advanced through the trans-septal catheter 504 to the left atrium 506, and down through the mitral valve leaflet 512 of mitral valve 508, and into the left ventricle 510. In some variations, the trans-septal sheath 504 has a steerable (or directional) tip to help guide the catheter(s) towards the leaflet and cardiac tissue beneath the leaflet. As can be seen in FIG. 5, mitral valve leaflet 512 is prolapsed so that it can no longer prevent the back flow of blood into the left atrium when the ventricle contracts.

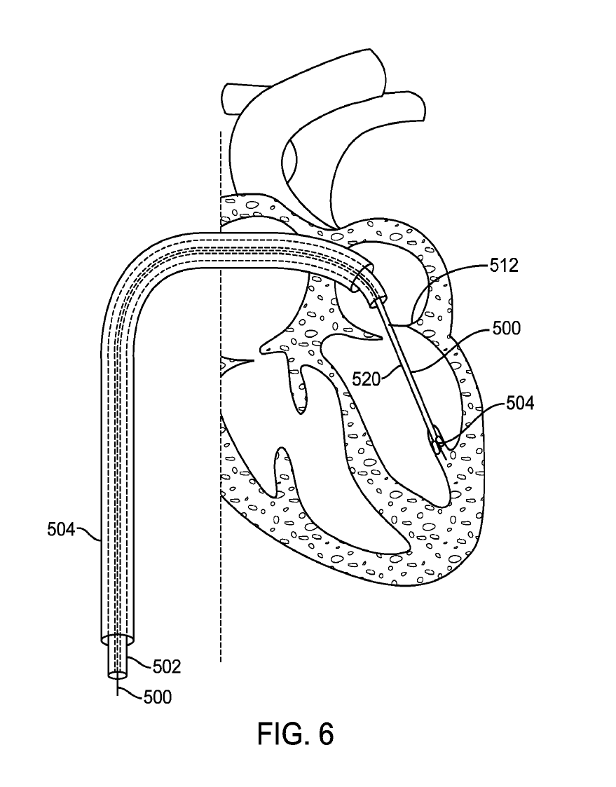

FIG. 6 shows synthetic chord device 520 being advanced to cardiac tissue located beneath prolapsed mitral valve leaflet 512, and specifically to the head of papillary muscle 504. In some variations, the catheter may be steerable (e.g., the tip may be at steerable in at least one direction). Thus, the trans-septal sheath 504 and the catheter 502 together may be used to guide the catheter 502. Once the distal end of the chord device 520 that includes the first reinforcing member is passed through the papillary muscle, the restraining wire 500 may be released from the chord device 520 and pulled back into delivery catheter 502, first back through the papillary muscle and then back through the mitral valve leaflet 512. Upon passage of the restraining wire from the mitral valve leaflet 512 back into the delivery catheter 502, the second reinforcing member deploys on the catheter side of leaflet to assume the unconstrained, securing configuration.

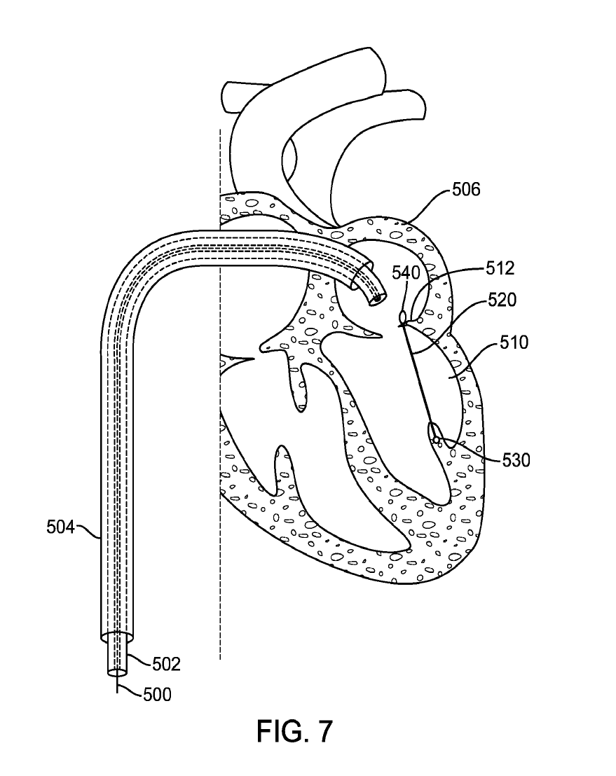

At this point the mitral valve and the papillary muscle are connected to each other, e.g., as depicted in FIG. 7. As shown in FIG. 7, chord 520 is securely connected to the papillary muscle by first deployed securing member 530 and is also securing connected to mitral valve leaflet 512 by second deployed securing member 540. In this manner, the ruptured valve has been repaired. The delivery catheter may then be removed, e.g., via convention protocols.

In addition to catheter based protocols, e.g., as illustrated in FIGS. 4 to 7, other minimally invasive protocols and devices may be employed. For example, endoscope protocols may be employed. In some instances, the transapical devices and methods as described in U.S. Pat. No. 7,635,386 and WO 2013/003228 are modified to deliver a synthetic chord device, e.g., as described above, where the in these publications of the devices and methods is specifically incorporated herein by reference. Additional non-catheter minimally invasive approaches of interest include those that access the heart through the chest using trocars and suitable minimally invasive instruments, either from the left or right side, e.g., using protocols adapted from those described in United States pending provisional application No. 61/800,570; the disclosure of which is herein incorporated by reference.

The subject methods also include the step of diagnosing a patient in need of cardiac valve repair, e.g., mitral valve repair. Primary mitral regurgitation is due to any disease process that affects the mitral valve device itself. The causes of primary mitral regurgitation include myxomatous degeneration of the mitral valve, infective endocarditis, collagen vascular diseases (e.g., SLE, Marfan's syndrome), rheumatic heart disease, ischemic heart disease/coronary artery disease, trauma balloon valvulotomy of the mitral valve, certain drugs (e.g. fenfluramine). If valve leaflets are prevented from fully coapting (i.e., closing) when the valve is closed, the valve leaflets will prolapse into the left atrium, which allows blood to flow from the left ventricle back into the left atrium, thereby causing mitral regurgitation.

The signs and symptoms associated with mitral regurgitation can include symptoms of decompensated congestive heart failure (e.g., shortness of breath, pulmonary edema, orthopnea, paroxysmal nocturnal dyspnea), as well as symptoms of low cardiac output (e.g., decreased exercise tolerance). Cardiovascular collapse with shock (cardiogenic shock) may be seen in individuals with acute mitral regurgitation due to papillary muscle rupture or rupture of a chorda tendineae. Individuals with chronic compensated mitral regurgitation may be asymptomatic, with a normal exercise tolerance and no evidence of heart failure. These individuals however may be sensitive to small shifts in their intravascular volume status, and are prone to develop volume overload (congestive heart failure).

Findings on clinical examination depend of the severity and duration of mitral regurgitation. The mitral component of the first heart sound is usually soft and is followed by a pansystolic murmur which is high pitched and may radiate to the axilla. Patients may also have a third heart sound. Patients with mitral valve prolapse often have a mid-to-late systolic click and a late systolic murmur.

Diagnostic tests include an electrocardiogram (EKG), which may show evidence of left atrial enlargement and left ventricular hypertrophy. Atrial fibrillation may also be noted on the EKG in individuals with chronic mitral regurgitation. The quantification of mitral regurgitation usually employs imaging studies such as echocardiography or magnetic resonance angiography of the heart. The chest x-ray in patients with chronic mitral regurgitation is characterized by enlargement of the left atrium and the left ventricle. The pulmonary vascular markings are typically normal, since pulmonary venous pressures are usually not significantly elevated. An echocardiogram, or ultrasound, is commonly used to confirm the diagnosis of mitral regurgitation. Color doppler flow on the transthoracic echocardiogram (TTE) will reveal a jet of blood flowing from the left ventricle into the left atrium during ventricular systole. Because of the difficulty in getting accurate images of the left atrium and the pulmonary veins on the transthoracic echocardiogram, a transesophageal echocardiogram (TEE) may be necessary to determine the severity of the mitral regurgitation in some cases. The severity of mitral regurgitation can be quantified by the percentage of the left ventricular stroke volume that regurgitates into the left atrium (the regurgitant fraction). Other methods that can be used to assess the regurgitant fraction in mitral regurgitation include cardiac catheterization, fast CT scan, and cardiac MRI.

Indications for surgery for chronic mitral regurgitation include signs of left ventricular dysfunction. These include an ejection fraction of less than 60 percent and a left ventricular end systolic dimension (LVESD) of greater than 45 mm.

### Kits

Also provided are kits that at least include the subject minimally invasive systems. The subject kits at least include a minimally invasive delivery device and a synthetic chord device of the subject invention, as well as instructions for how to use the synthetic chord device in a procedure. In some embodiments, the kits can include a set of two or more synthetic chord devices. In other embodiments, a set of synthetic chord devices can include at least three synthetic chord devices, e.g., four or more, five or more, six or more, etc. Where desired, the delivery device may be pre-loaded with the chord.

In some embodiments, a set of synthetic chord devices includes two or more synthetic chord devices in which at least two of the synthetic chord devices have flexible connectors (e.g., first flexible connectors and/or one or more first flexible connectors and/or one or more second flexible connectors) of different lengths. In other embodiments, the flexible connector (e.g., first flexible connector) portions of the synthetic chord devices are all of differing lengths. In some embodiments, a set of synthetic chord devices can have two or more synthetic chord devices in which the flexible connectors (e.g., first flexible connectors) are of the same length. A set of synthetic chord devices can therefore have two or more some synthetic chord devices in which some are of the same length, and some are of a different length. For example, in one embodiment a set of six synthetic chord devices can have two synthetic chord devices in which the flexible connector (e.g., first flexible connector) portion is 8 mm in length; two synthetic chord devices in which the flexible connector portion is 10 mm in length; and two synthetic chord devices in which the flexible connector portion is 12 mm in length. In another embodiment, a set of synthetic chord devices can have four synthetic chord devices in which the flexible connector (e.g., first flexible connector) in all of them is 10 mm in length.

In addition, in some embodiments, the synthetic chord devices can be color-coded, such that a desired length of the synthetic mitral valve chord, or flexible connector (e.g., first flexible connector) element, can be easily determined. For example, a package with multiple synthetic chord devices can have flexible connectors (e.g., first flexible connectors) of two different colors arranged in an alternating pattern to allow a medical practitioner (e.g., scrub nurse) to readily distinguish one synthetic chord device from another. For example, a set of ten synthetic chord devices in a kit can be arranged in two horizontal rows of five in each row. An exemplary arrangement of associated flexible connector colors would be, in the top row: white, green, white, green, white, and in the bottom row: green, white, green, white, green. Further details of packaging that can be adapted for use with the synthetic chord devices of the subject invention are disclosed in U.S. Pat. No. 6,029,806, incorporated herein by reference. In this manner, a scrub nurse can readily associate each tissue piercing member (e.g., needle) with the synthetic chord device containing the correct length of synthetic mitral valve chord, or flexible connector. By color coding the synthetic chord devices with alternating, contrasting flexible connector colors, more synthetic chord devices can be stored in a package of a given size without causing confusion. The needle associated with each synthetic chord device can be sufficiently separated from other such needles to allow grasping of each needle with a needle holder, while maintaining identification of the needle as belonging to the same synthetic chord device.

The kit can also include a measuring tool, which can be disposable, for determining a desired length of a synthetic chord by measuring a desired distance, such as the distance between a prolapsed cardiac valve leaflet and cardiac tissue located below the prolapsed cardiac valve leaflet. Such a measuring tool may include, but is not limited to any suitable measuring device, such as a caliper, a Mohr Suture Ruler Device™ (Geister, Tuttlingen, Germany), or sterile disposable flexible tape measure.

Components of the kits may be present in separate containers, or multiple components may be present in a single container. For example, the delivery device and chord(s) may be present in different containers, or combined in a single container (such as where the delivery device is preloaded with the chord), as desired. In some instances, the container(s) are sterile packaging containers.

The instructions for using the devices as discussed above are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

Notwithstanding the appended clauses, the disclosure is also defined by the following clauses:

1. A percutaneous or minimally invasive system comprising:

(a) a synthetic chord comprising:
(i) a flexible connector comprising a first end and a second end;
(ii) a first tissue securing member located at the first end of the flexible connector, wherein the first tissue securing member transitions from a linear to a planar configuration upon deployment; and
(iii) a second tissue securing member located at the second end of the flexible connector, wherein the second tissue securing member transitions from a linear to a planar configuration upon deployment; and

(b) a catheter-based minimally invasive delivery device configured to deliver the synthetic chord to an internal body location.

2. The system according to Clause 1, wherein the linear configuration of the first tissue securing member is one that lacks a secondary structure and the planar configuration is one that has a secondary structure.
3. The system according to Clause 2, wherein the secondary structure is a planar spiral configuration.
4. The system according to Clauses 1, 2 or 3, wherein the end of the first tissue securing member distal to the flexible connector is configured to pierce tissue.
5. The system according to Clause 1, wherein the linear configuration of the second tissue securing member is one that lacks a secondary structure and the planar configuration is one that has a secondary structure.
6. The system according to Clause 5, wherein the secondary structure is a planar spiral configuration.
7. The system according to Clause 1, wherein the linear configuration of the second tissue securing member comprises the second securing member having a longitudinal axis at least substantially parallel to the longitudinal axis of the flexible connector and the planar configuration comprises the second securing member having a longitudinal axis at least substantially perpendicular to the longitudinal axis of the flexible connector.
8. The system according to Clause 7, wherein the second securing member is a bar.
9. The system according to Clause 8, wherein the bar comprises stainless steel.
10. The system according to any of clauses 1 to 8, wherein at least one of the first and second securing members comprises a shape memory material.
11. The system according to Clause 10, wherein the shape memory material is a metal alloy.
12. The system according to Clause 11, wherein the metal alloy comprises a nickel alloy.
13. The system according to Clause 12, wherein the nickel alloy is a nickel-titanium alloy.
14. The system according to Clause 11, wherein the nickel alloy is a chromium-cobalt-nickel alloy.
15. The system according to any of the preceding clauses, wherein the flexible connector comprises a polymer.
16. The system according to Clause 15, wherein the polymer comprises expanded PTFE (ePTFE).
17. The system according to any of the preceding clauses, wherein the flexible connector has a length ranging from 5 mm to 100 mm.
18. The system according to any of the preceding clauses, wherein the minimally invasive delivery device comprises a catheter.
19. The system according to clause 18, wherein the synthetic chord is positioned at the distal end of the catheter.
20. The system according to Clause 19, wherein the catheter is a steerable catheter.
21. A percutaneous or minimally invasive method for connecting a first tissue to a second tissue, the method comprising:

(a) positioning the distal end of a minimally invasive or percutaneous system at a target tissue location comprising the first and second tissue via minimally invasive protocol, wherein the minimally invasive system comprises a synthetic chord comprising:
(i) a flexible connector comprising a first end and a second end; and
(ii) first and second tissue securing members respectively located at the first and second ends of the flexible

connector, wherein the first and second tissue securing

members each transition from a linear to a planar

configuration upon deployment; and

(b) deploying the synthetic chord from the minimally invasive system in a manner sufficient to connect the first and second tissue.

22. The method according to Clause 21, wherein the target tissue location comprises a cardiac location.
23. The method according to Clause 22, wherein the first tissue comprises a papillary muscle.
24. The method according to Clause 23, wherein the second tissue comprises a cardiac valve leaflet.
25. The method according to Clause 24, wherein the cardiac valve leaflet comprises a mitral valve leaflet.
26. The method according to any of Clauses 21 to 25, wherein the minimally invasive protocol comprises delivering the distal end of the minimally invasive system through the is vasculature.
27. The method according to Clause 26, wherein the minimally invasive protocol comprises accessing the vasculature at a site distal to the target tissue location.
28. The method according to Clause 27, wherein the method comprises accessing the vasculature at a femoral vessels site.
29. The method according to any of Clauses 21 to 28, wherein the deploying comprises sequentially moving the first securing member out of the minimally invasive system so that the securing member transitions to the planar configuration to secure the flexible connector to the first tissue and then moving the second securing member out of the minimally invasive system so that the second securing member transitions to the planar configuration to secure the flexible connector to the second tissue and connect the first and second tissues.
30. The method according to Clause 29, wherein the method comprises passing the distal end of the minimally invasive system through the second tissue to locate the distal end at the first tissue prior to deployment of the synthetic chord.
31. The method according to any of Clauses 21 to 30, wherein the minimally invasive system is a system according to any of Clauses 1 to 20.
32. A kit comprising:

(a) a set of one or more synthetic chord devices, each device of said set comprising:
(i) a flexible connector comprising a first end and a second end; and
(ii) first and second tissue securing members respectively located at the first and second ends of the flexible connector, wherein the first and second tissue securing members each transition from a linear to a planar configuration upon deployment; and

(b) a minimally invasive delivery device configured to deliver a synthetic chord to an internal body location.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A synthetic chord comprising:
(a) a first flexible connector comprising a first end and a second end;
(b) an attachment element comprising a tissue piercing member and a securing member located at the first end of the flexible connector, wherein the securing member transitions from a linear to a planar configuration upon separation of the tissue piercing member from the attachment element; and
(c) a reinforcing element located at a second end of the flexible connector.

2. The synthetic chord device according to claim 1, wherein the linear configuration is one that lacks a secondary structure and the planar configuration is one that has a secondary structure.

3. The synthetic chord device according to claim 2, wherein the secondary structure is a spiral configuration.

4. The synthetic chord device according to claim 3, wherein the spiral configuration has 1.5 turns.

5. The synthetic chord device according to claim 3, wherein the securing member includes a planar maintenance structure.

6. The synthetic chord device according to claim 1, wherein the linear configuration comprises the securing member having a longitudinal axis at least substantially parallel to the longitudinal axis of the flexible connector and the planar configuration comprises the securing member having a longitudinal axis at least substantially perpendicular to the longitudinal axis of the flexible connector.

7. The synthetic chord device according to claim 6, wherein the securing member is a bar.

8. The synthetic chord device according to claim 1, wherein the securing member comprises a shape memory material.

9. The synthetic chord device according to claim 1, wherein the securing member and tissue piercing member of the attachment element are separated from each other by a second flexible connector.

10. The synthetic chord device according to claim 1, wherein the tissue piercing member and securing member are operably connected to each other by an interlocking member.

11. The synthetic chord device according to claim 1, wherein the reinforcing element is a pledget.

12. The synthetic chord device according to claim 1, wherein the device comprises a third flexible member attached to the reinforcing element at a first end and a second attachment element at a second end, wherein the second attachment element comprises a tissue piercing member and a securing member that transitions from a linear to a planar configuration upon separation of the tissue piercing member from the attachment element.

13. The synthetic chord device according to claim 12, wherein the first and third flexible members form a continuous flexible structure.

14. A method for connecting a first tissue to a second tissue, the method comprising:
(a) passing a tissue piercing member of a synthetic chord device according to claim 1 through the first tissue so that the reinforcing element contacts the first tissue;
(b) passing the tissue piercing member through the second tissue; and
(c) separating the tissue piercing member from the attachment element to transition the securing element to a planar configuration and connect the first tissue to the second tissue.

15. A kit comprising:
a set of two or more synthetic chord devices according to claim 1.
